(19) United States
(12) Patent Application Publication
(10) Pub. No.: US 2020/0008394 A1
(43) Pub. Date: Jan. 9, 2020

(54) PET HEALTHCARE APPARATUS WITH BACTERIOSTATIC AND STERILIZATION SYSTEM

(71) Applicant: avec Co., Ltd., Seoul (KR)

(72) Inventor: Kyung Ho CHUN, Yongin-si (KR)

(21) Appl. No.: 16/268,452

(22) Filed: Feb. 5, 2019

(30) Foreign Application Priority Data
Jul. 9, 2018 (KR) 10-2018-0079659

(51) Int. Cl. A01K 13/00; B01D 46/00; B01D 46/10; A61L 9/20

(52) U.S. Cl. CPC A01K 13/001; B01D 46/0038; B01D 46/10; A61L 2209/14; A61L 9/20; A61L 2209/12; B01D 46/0023

(57) ABSTRACT

The present invention relates to a pet healthcare apparatus with a bacteriostatic and sterilization system, and more particularly, to a pet healthcare apparatus with a bacteriostatic and sterilization system, in which air passes through five stages including a discharge and suction part provided with antibacterial materials, a fan filter, a HEPA filter, an UV-C LED lamp, and a copper-coated fin heater, thereby taking care of pet's skin and removing, in an efficient way, various fine dust on the hairs contaminated due to external air, dust mites, fungal spores and dead hairs during molting.

### CROSS-REFERENCE TO RELATED APPLICATIONS

This patent application claims priority under 35 U.S.C. § 119 to Korean Patent Application No. 10-2018-0079659 filed on Jul. 9, 2018, the entire contents of which are incorporated by reference herein.

### TECHNICAL FIELD

The present invention relates to a pet healthcare apparatus with a bacteriostatic and sterilization system, and more particularly, to a pet healthcare apparatus with a bacteriostatic and sterilization system, in which air passes through five stages including a discharge and suction part provided with antibacterial materials, a fan filter, a HEPA filter, a UV-C LED lamp, and a copper-coated fin heater, thereby taking care of pet's skin and removing, in an efficient way, various fine dust on the hairs contaminated due to external air, dust mites, fungal spores and dead hairs during molting.

### BACKGROUND

Recent increase in single family household and aging in the population contributes to the increase in the families raising pets, such as dogs and cats, to be emotionally comforted by the communication with the pets.

Pets are residing together inside a single family house or an apartment. As the pets go around every corner of the house, dust on hairs contaminates the internal environment of the house while wallowing in a bed, sofa or carpet, or nestling in an arm, resulting in respiratory diseases.

In order to maintain the pets sterile, pets may be bathed in a Pet Shop or an individual bathes pets in person. However, wet pet hairs which are not completely dried after bathing may result in cold or skin diseases.

If a hair dryer is used to dry a pet placed in a cradle, it is unavailable to capture pet's hairs missing during drying. In addition, it takes time to dry the pet, which results in stress to the pets. In order to solve these problems, a pet dry room has been developed and come into wide use to dry pet hairs.

Korean Patent No. 10-1840655 (registered on Mar. 15, 2018) discloses an exemplary pet dry room.

Disclosed is a pet dry room including a body formed with an accommodating space to accommodate a pet, a door connected to the front surface of the body and provided to open and close the accommodating space, an air circulating module provided with a motor, a fan, a flow passage, a nozzle and a heater to discharge air to the accommodating space in which air flows. The air flowing in through air blowing holes formed in an air blowing tray, which is also used for a defecation plate formed on the bottom surface of the accommodating space, and through nozzles formed at the lateral and rear surfaces of accommodating space is suctioned to the first suction passage disposed in the front side of the ceiling surface of the accommodating space.

However, there is a problem that air blown from the nozzles formed at lateral and rear surfaces dries eyes and nose of the pet due to the air blown directly to facial parts such as a nose, ears and eyes and that it takes a long time to dry the belly or legs.

### SUMMARY

The purpose of the present invention is to provide a pet healthcare apparatus with a bacteriostatic and sterilization system, in which air passes through five stages including a discharge and suction part provided with antibacterial materials, a fan filter, a HEPA filter, an UV-C LED lamp, and a copper-coated fin heater, thereby taking care of pet's skin and removing, in an efficient way, various fine dust on the hairs contaminated due to external air, dust mites, fungi and dead hairs during molting.

In addition, another purpose of the present invention is to provide a pet healthcare apparatus with a bacteriostatic and sterilization system, allowing the wet hairs of a pet having double hair to be easily dried by forming an air flow passage at the lower surface and left and right sides thereof, and allowing efficient moisture and fine dust removal by allowing air to deeply penetrate between the hairs.

In addition, another purpose of the present invention is to provide a pet healthcare apparatus with a bacteriostatic and sterilization system, in which discharge parts provided at both lateral surfaces of an inner accommodating space of the pet healthcare apparatus at a double angle such that the air can deeply penetrate between the hairs even when the pet is sitting or lying down, thereby enabling effective drying.

To achieve the above purposes, the pet healthcare apparatus (1) of the present invention, in which an accommodating space (S) accommodating a pet is formed, has a care device (10) provided therein, wherein the care device (10) comprises: a lower device (100) comprising a front member (120), a rear member (130), and a lateral member (140); a bottom surface (50) coupled to the upper portion of the lower device (100) and having bottom lateral grooves (57) formed at both lateral surfaces thereof; a discharge part (200) coupled to the upper portion of the bottom lateral grooves (57) of the bottom surface (50) such that purified air is discharged toward the accommodating space (S); and a suction part (300) suctioning air discharged from the discharge part (200) and discharging the suctioned air to a case space part (111) inside the lower device.

In addition, the suction part (300) is injected by using an antibacterial material, and comprises a filter (P) collecting the hairs of a pet and dust, a HEPA filter (H) collecting and antibacterializing dust, fine dust, fungal spores, and bacteria, and a UV-C LED lamp (R) having a sterilizing function, and the lower device is provided with copper coating fin heaters (180, 180′).

In addition, in the discharge part (200), a first discharge hole (220) and a second discharge hole (230) are formed, wherein the first discharge hole (220) and the second discharge hole (230) are formed at a double angle of 5° and 13°.

In addition, the first discharge hole (220) and the second discharge hole (230), which are formed at a double angle of 5° and 13°, are provided such that air is discharged toward the bottom plate at any one angle of the double angle, and air is discharged in the lateral direction of the accommodating space at the other angle thereof.

In addition, the lower device comprises a fan (160), a first guide (170), and a second guide (171), wherein when air suctioned to the upper portion of the fan (160) is discharged to a fan discharge part (161) of the fan (160), the air is discharged toward an air flow passage formed between the first guide (170) and the second guide (171).

In addition, the rotary air, rotating in one direction, is formed by the fan provided inside the lower device, and when the rotary air formed by the fan is being discharged to the accommodating space through the discharge part, the rotary air is accelerated by the first discharge hole (220) and the second discharge hole (230), provided in the discharge part, and is discharged to the accommodating space.

In addition, at the front surface of the front member (120), a front suction hole (121) for allowing the air outside the pet healthcare apparatus to flow into the case space part (111) is formed.

In addition, the suction part comprises a suction body (310), a through-hole (311) formed at the upper portion of the suction body, a through-groove cover (312) formed at the through-hole, a filter (P) mounted at the through-hole, a HEPA filter (H) mounted to a discharge hole (330) of the suction part, and a UV-C LED lamp (R) mounted at the rear surface of the HEPA filter.

In addition, at the lower portion of the suction part (300), a suction protrusion part (320) coupled to a rear coupling groove (131) of the rear member (130) is formed, such that the air suctioned by the suction part (300) is discharged to the case space part (111) through the suction protrusion part (320).

In addition, inside the suction part (300), a dust collector collecting and removing solid or liquid particles suspended in gas is mounted.

In addition, inside the suction part (300), an air purifier having a plasma wave function for generating oxygen ions is mounted.

In addition, inside the suction part (300), a deodorizing filter for removing smell and odors from the inside of the pet healthcare apparatus is mounted.

The purpose of the invention is to allow air to pass through five stages including a discharge and suction part provided with antibacterial materials, a fan filter, a HEPA filter, an UV-C LED lamp, and a copper-coated fin heater, thereby taking care of pet's skin and removing, in an efficient way, various fine dust on the hairs contaminated due to external air, dust mites, fungal spores and dead hairs during molting.

In addition, the present invention is effective in easily drying the wet hairs of a pet having double hair by forming an air flow passage at the lower surface and left and right sides of the pet healthcare apparatus and in efficiently removing moisture and fine dust by allowing air to deeply penetrate between the hairs.

In addition, by forming the discharge part provided at both lateral surfaces of the inner accommodating space of the pet healthcare apparatus at a double angle, air can deeply penetrate between the hairs even when the pet is sitting or lying down, thereby enabling effective drying.

### BRIEF DESCRIPTION OF THE DRAWINGS

FIG. 1 is a perspective view showing the pet healthcare device coupled inside a healthcare apparatus.

FIG. 2 is a perspective view of the pet healthcare apparatus with a bacteriostatic and sterilization system.

FIG. 3 is an exploded perspective view of the healthcare apparatus according to the present invention.

FIG. 4 is an embodiment of the healthcare apparatus in which air is circulated.

FIG. 5 is a bottom view of the healthcare apparatus according to the present invention.

FIG. 6 is a perspective view of the discharge part according to the present invention.

FIG. 7 is a perspective view of a suction part according to the present invention.

### DETAILED DESCRIPTION OF THE DISCLOSURE

The terms and words used in the present description and claims should not be interpreted in a conventional or dictionary sense, and should be interpreted, on the basis of the principle that the inventor can properly define the concept of a term in order to describe the invention in the best way possible, in meanings and concepts consistent with the technical idea of the present invention.

Therefore, the embodiments described in the present description and drawings are only the most preferred embodiments of the present invention and are not intended to represent all the technical ideas of the present invention. Therefore, it should be understood that various equivalents and modifications may be substituted for the embodiments at the time of filing of the present application.

Before describing the present invention with reference to the accompanying drawings, it should be noted that the present invention is not described or specifically described with respect to a matter which is not required to reveal the gist of the present invention, i.e. a known feature that can be easily added by a person skilled in the art.

The present invention relates to a pet healthcare apparatus with a bacteriostatic and sterilization system.

The pet healthcare apparatus (1) has an accommodating space (S) accommodating a pet, formed therein,

wherein the pet healthcare apparatus (1) has a care device (10) provided therein,

wherein the pet healthcare apparatus with a bacteriostatic and sterilization system is characterized in that the care device (10) comprises: a lower device (100) comprising a front member (120), a rear member (130), and a lateral member (140); a bottom surface (50) coupled to the upper portion of the lower device (100) and having bottom lateral grooves (57) formed at both lateral surfaces thereof; a discharge part (200) coupled to the upper portion of the bottom lateral grooves (57) of the bottom surface (50) such that purified air is discharged toward the accommodating space (S); and a suction part (300) suctioning air discharged from the discharge part (200) and discharging the suctioned air to a case space part (111) inside the lower device.

The present invention will be described in detail below with reference to the accompanying drawings.

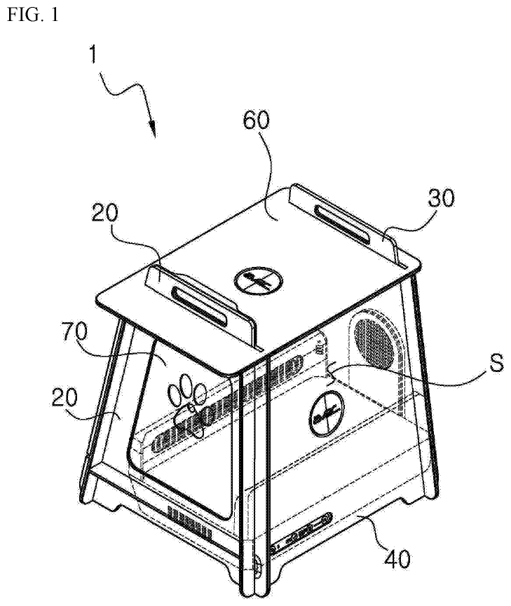

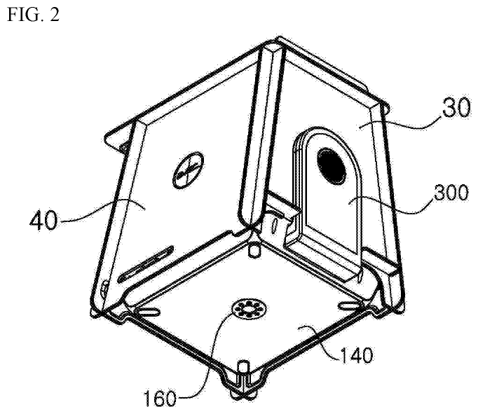

FIG. 1 is a perspective view of a healthcare device according to the present invention coupled to a pet healthcare apparatus, and FIG. 2 is a perspective view of a pet healthcare apparatus with a bacteriostatic and sterilization system according to the present invention.

Referring to FIGS. 1 and 2, the pet healthcare apparatus 1 includes an accommodating space (S) for accommodating a pet.

The accommodating space (S) includes a front plate (20) formed at the front side (20) of the accommodating space (S), a rear plate (30) corresponding to the front plate (20), a lateral plate (40) at both sides between the front plate (20) and the rear plate (30), a bottom plate (50) at the lower side of the accommodating space (S), and a ceiling plate (60) at the upper portion corresponding to the bottom plate (50).

A through-hole (31) is formed at the center of the rear plate (30) such that a suction part (300), described below, is coupled, and any one lateral plate (40) among both lateral plates (40) is provided with a lateral control through-hole (44) to which a controller (141) can be coupled.

In addition, at both sides of the bottom plate (50), a bottom side groove (57), penetrating therethrough, is formed.

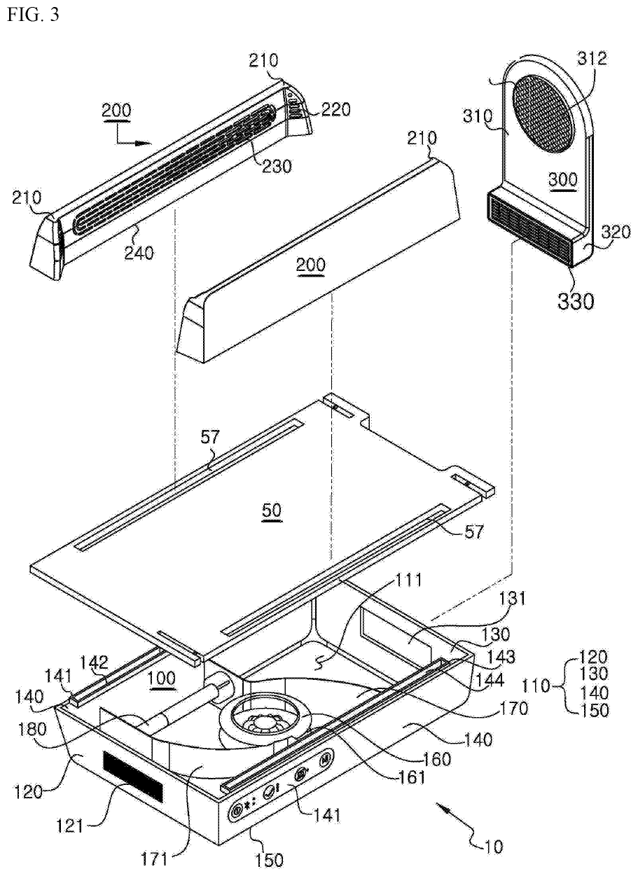

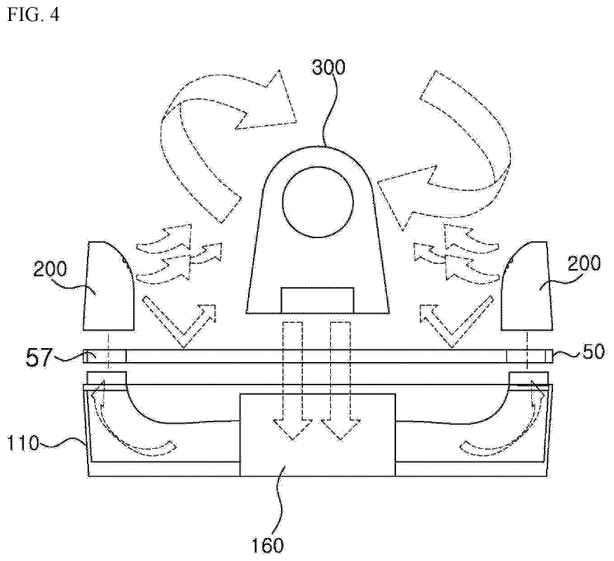

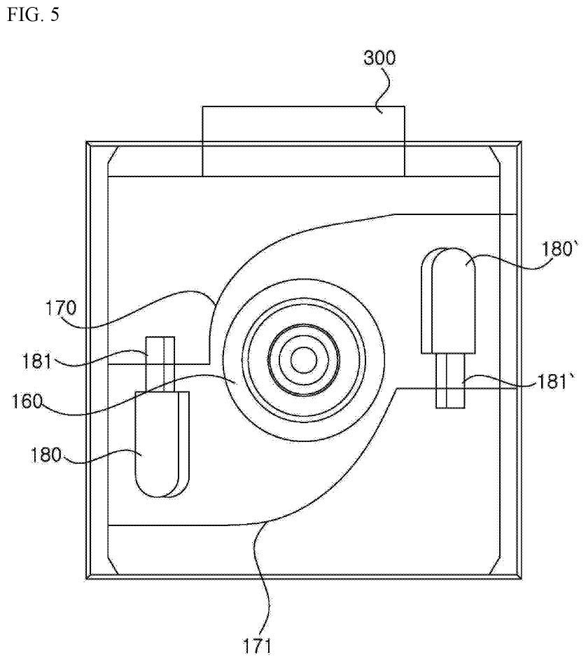

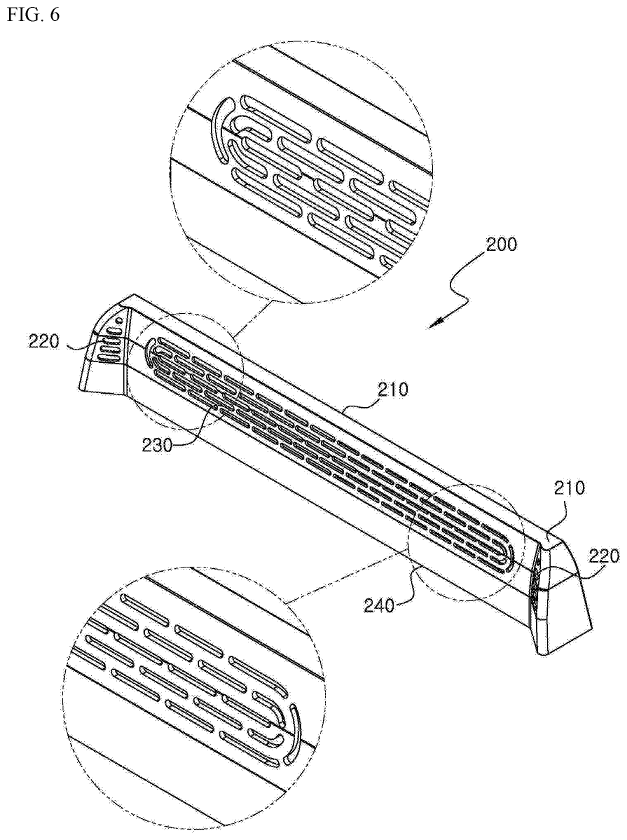

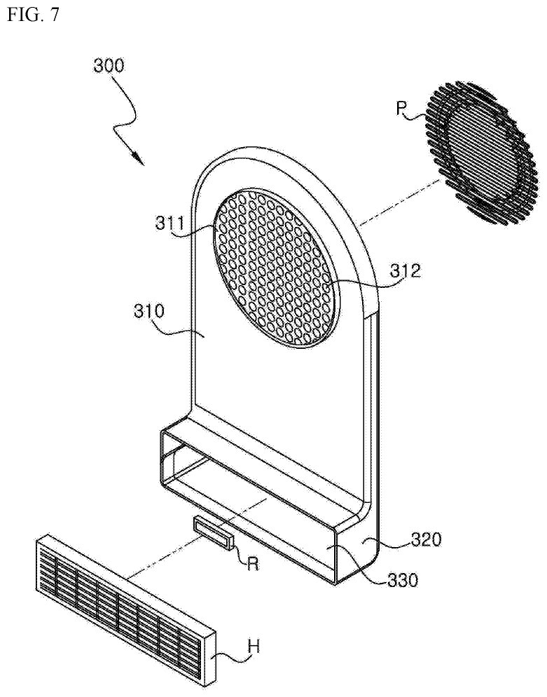

FIG. 3 is an exploded perspective view of the healthcare apparatus according to the present invention, FIG. 4 is an embodiment in which air is circulated in the healthcare apparatus according to the present invention, FIG. 5 is a bottom view of the healthcare apparatus according to the present invention, FIG. 6 is a perspective view of the discharge part according to the present invention, and FIG. 7 is a perspective view of the suction part according to the present invention.

Referring to FIGS. 3 to 7, the healthcare device (10) is provided in the lower portion of the inside of the pet healthcare apparatus (1).

The healthcare apparatus (10) comprises the lower device (100), the bottom plate (50), the discharge part (200), and the suction part (300).

The lower device (100) is composed of the case (110) of which the upper portion is open and in which the case space part (111) is formed.

The case (110) is provided with the front member (120) at the front side of the case space part (111), and at one side of the front member (120), a front suction hole (121) formed of a plurality of through-holes such that external air can flow to the inside of the case space part (111).

At the rear side of the case space part (111), corresponding to the front member (120), the rear member (130) is formed, wherein a rear coupling groove (131) is formed at the center of the rear member (130), and the discharge hole (330) of the suction hole (300), described below, is coupled to the rear coupling groove (131).

In addition, inside the suction part (300), the dust collector collecting and removing solid or liquid particles suspended in gas can be mounted.

In addition, inside the suction part (300), the air purifier having a plasma wave function for generating oxygen ions can be mounted.

In addition, inside the suction part (300), the deodorizing filter for removing smell and odors from the inside of the pet healthcare apparatus can be mounted.

In addition, the lateral member (140) is formed at both sides between the rear member (130) and the front member (120).

For the lateral member (140), the controller (141) allowing a user to operate the healthcare device (10), is formed at one outer side, wherein the controller (141) is formed at any one lateral member (140) among the lateral members (140).

In addition, the lateral member (140) is provided with the lateral discharge hole (142), protruding upward, at the inner side thereof in the direction of the case space part (111), wherein the lateral discharge hole (142) can have the same width as the distance between the first guide (170) and the second guide (171) described below.

In addition, the lateral discharge hole (142) is provided with the lateral through-groove (143) formed at the inner upper portion thereof so as to penetrate to the bottom, and has a flow passage shape through which air is discharged to the lateral through-groove (143) by the fan (160) described below.

At the lower side of the case space part (111), the lower member (150) is formed.

At the center of the lower member (150), the fan (160) described below, suctioning air discharged toward the space part (111) through the discharge hole (330) of the suction part (300) described below and discharge the same to the lateral through-groove (143) of the lateral discharge hole (142). The lateral surface of the fan (160) is provided with the fan discharge part (161) having an open shape.

The fan (160) suctions air by the upper portion thereof and discharges the suctioned air through the fan discharge part (161) formed at the lateral surface of the fan (160).

At this time, the air discharged from the fan discharge part (161) is discharged to the air flow passage formed between the first guide (170) and the second guide (171) described below.

The first guide (170) is formed in a “ [symbol] ” shape around the fan (160) in the direction of the rear member (130), and the second guide (171) is formed in a “ [symbol] ” shape at a front plate (20) side corresponding to the first guide (170).

The first guide (170) and the second guide (171) are coupled to the upper portion inside the bottom plate (50) such that the air discharged to the fan discharge part (161) of the fan coupled to the center of the bottom plate (50) is prevented from being discharged in all directions with respect to the case space part (111), and the air is discharged toward the air flow passage formed between the first guide (170) and the second guide (171).

At this time, the air flow passage formed between the first guide (170) and the second guide (171) has the same width as the air flow passage of the lateral discharge hole (142) provided in the lateral member (140).

The air flow passage formed between the first guide (170) and the second guide (171) can be provided with copper-coated fin heaters (180, 180′). In addition, one connection part (181, 181′) can be provided at each end portion of the copper-coated fin heaters (180, 180′).

For these copper-coated fin heaters (180, 180′), the connection part (181) of the copper coated fin heater (180) having a “U” shape penetrates one flat surface of the first guide (170) formed on the lateral plate (40) around the fan (160) and can be fastened through a bolt.

Accordingly, the copper-coated fin heaters (180, 180′) are respectively coupled and fixed to flat one surfaces of the first guide (170) and the second guide (171) around the fan (160). These copper-coated fin heaters (180, 180′) emit heat so as to serve to sterilize air and to heat the air to a temperature most suitable for pets.

At the upper portion of the lower device (100), the bottom plate (50) having a plate shape, at which a pet can be seated is coupled. At both lateral surfaces of this bottom plate (50), the bottom lateral groove (57) is provided, wherein the bottom lateral groove (57) is provided such that the lateral discharge hole (142) of the lateral member (140) penetrates and is coupled to the same.

At this time, the upper portion of the lateral discharge hole (142) of the lateral member (140) can have shape further protruding than the upper surface of the bottom plate (50). In addition, at the bottom lateral groove (57) of the bottom plate (50), respective discharge parts (200) are provided.

This discharge part (200) is provided at both sides of the bottom plate (50) and is provided with a curved protrusion part (210) having a long shape toward the front plate (20) and the rear plate (30) and having curved toward the accommodating space (S). At the curved protrusion part (210), the plurality of first discharge holes (220) are respectively formed, and the second discharge hole (230) having the plurality of through-holes is formed between the curved protrusion parts (210).

At this time, the second discharge hole (230) of the discharge part (200) provided at both lateral surfaces has a shape in which the width of the through-hole increases as going toward the rear plate (30) from the front plate (20), and the second discharge hole (230) of the discharge part (200) at the other side has a shape in which the width of the through-hole increases as going toward the front plate (20) toward the rear plate (30).

In other words, the second discharge hole (230) has a laterally symmetrical structure such that the air discharged to the accommodating space through the discharge hole is changed into rotary air in the accommodating space.

A hollow is formed inside the first discharge hole (220) and the second discharge hole (230) of the discharge part (200), and a through-hole (240), having the same size as the bottom lateral groove (57) of the bottom plate (50), is formed at the lower portion of the discharge part (200) so as to be communicated with the hollow of the discharge part (200).

In addition, the through-hole (240) can be coupled to the lateral discharge hole (142) of the lateral member (140) penetrating the bottom lateral groove (57) of the bottom plate (50).

In addition, at the hollow inside the discharge part (200), a plurality of ultraviolet LED mood lamps emitting light having a wavelength of 405 nm toward the accommodating space (S) and IR LED lamps, wherein the plurality of ultraviolet LED mood lamps and IR LED lamps are alternately connected in series.

This ultraviolet LED mood lamp emitting light having a wavelength of 405 nm forms an atmosphere for the pets to have psychological stability and serves as a sterilizer, and the IR LED lamp serves as deodorizer and drier.

In addition, these first discharge hole (220) and second discharge hole (230) have an inclination angle of 5° and 13°.

In addition, the first discharge hole (220) and the second discharge hole (230) are provided such that air is discharged at any one angle of the double angle toward the bottom plate, and air is discharged at the other angle in the lateral direction of the accommodating space.

Accordingly, the first discharge hole (220) and the second discharge hole (230) allows the air blown toward the bottom plate (50) and the accommodating space (S) to cause rotary air such that foreign substances can be efficiently removed from the hairs of a pet having double hair, the wet hairs of a pet can be quickly dried, and the air is prevented from being blown to the eyes, noses, and the like of a pet, thereby preventing the eyes, noses, and the like of the pet from being dried.

In addition, the air blown through the discharge part is discharged, when the air in the accommodating space (S) is contaminated by foreign substances, ultrafine dust, pet hair, and mold and the contaminated air is suctioned through the suction part (300) and is purified, to the accommodating space (S).

The suction part is injected by containing an antibacterial material, is provided at the rear member (130) of the case (100), and comprises a suction body (310) formed above the bottom plate (50), wherein the suction body (310) comprises the through-hole, penetrating in a front-rear direction, formed at the center thereof, and the filter (P) provided in a free filter form inside the through-hole (311).

The filter (P) collects dust mites and fungal spores included in pet hair and in the air, and the dust mites and fungal spores collected in the filter (P) can be cleanly washed off by water, thereby facilitating the management thereof.

In addition, the through-groove cover (312) is provided at the through-hole (311), and the through-groove cover (312) serves to fix the filter (P) not to be taken out of the suction hole. At this time, the plurality of grooves are formed in the through-groove cover (312) such that air can easily flow to the filter.

In addition, at the lower portion of the suction body (310) of the suction part (300), a suction protrusion part (320) protruding toward the case space part (111) is formed, wherein one protruding end of the suction protrusion part (320) is coupled to the rear coupling groove (131) of the rear member (130), the discharge hole (330) is formed at the end portion of the suction protrusion part (320) such that the air having been suctioned through the suction part (300) is discharged to the case space part (111), and the UV-C LED lamp (R) and the HEPA filter (H) are coupled to the discharge hole (330) in order.

The UV-C LED lamp (R) functions as a sterilizing lamp for a UV LED medical device, and the HEPA filter (H) filters ultrafine dust, fungus spores, and the like which are not filtered by a filter (P) and collects dust, fungus spores, and bacteria. The antibacterializing and sterilization are performed by the UV-C LED lamp (R).

These filter (P), UV-C LED lamp (R), and the HEPA filter (H) are well-known technologies, and will not be described in detail.

The healthcare device (10) as described above may be configured such that the foreign substances, dust, ultrafine dust, bacteria, and the like adhering to the hair of a pet do not stay in the accommodating space (S) of the pet healthcare apparatus (1) and are purified through five-stage care of the discharge part (200) and the suction part (300) containing an antibacterial material, the filer (P), the HEPA filter (H), the UV-C LED lamp (R), and the copper-coated fin heaters (180, 180′), thereby maintaining and managing the accommodating space (S) in which a pet is accommodated in pleasant state.

More specifically, a first stage care is a step exhibiting an antibacterial effect by the antibacterial material included the discharge part (200) and the suction part (300) at the timing of the manufacture thereof.

In addition, a second stage care is a step in which a free filter is coupled to the inside of the suction part (300) so as to collect the dust mites and fungal spores of the accommodating space (S).

In addition, a third stage care is a step in which fine dust, fungal spores, and bacteria, which are not removed in the second step, are collected through the HEPA filter (H) coated with photocatalyst.

In addition, a fourth stage care is a step of antibacterializing and sterilizing bacteria included in the air through the UV-C LED lamp.

In addition, a fifth stage care is a step of further sterilizing the air purified through the first to fourth steps for the bacteria included in the air through the copper-coated fin heaters (180, 180′), and purifying the air, and heating the air to an appropriate temperature.

The healthcare device (10) coupled to the lower side of the pet healthcare apparatus (1) allows purified air to circulate and be supplied to the inner accommodating space of the pet healthcare apparatus (1), thereby efficiently removing various fine dust, dust mites, fungal spores adhering to the hair of a pet and dead hairs during molting.

In other words, five-stage pet healthcare is achieved, for removing foreign substances such as dust adhering to the hairs of a pet, who has wandered around a house or has taken a walk, or removing and drying moisture from the hairs of a pet after bathing, when a user remotely or manually operates the controller (141), formed at the lateral member (140) of the lower device (100), after making the contaminated pet accommodated in the accommodating space (S) of the pet healthcare apparatus (1).

At this time, by the operation of the fan (160) of the lower device (100), external air can flow into front suction hole (121), formed at the front member (120) of the lower device (100).

This is to allow fresh external air to flow into the inside of the pet healthcare apparatus (1) so as to supply fresh air to the pet accommodated in the accommodating space (S).

In addition, the ultraviolet LED mood lamp emitting light having a wavelength of 405 nm and the IR LED lamp, which are provided in the hollow inside the discharge part (200) form an atmosphere for the pets to have psychological stability, additionally sterilize and deodorize air, and removes moisture in the air.

In addition, at the lower portion of the pet healthcare apparatus (1), rotary air, rotated in one direction by the fan (160) and the flow passage, which are provided in the lower device (10) is formed, and the formed rotary air is accelerated by the first discharge hole (220) and the second discharge hole (230), provided in the discharge part (200), while being discharged by the discharge part (200) so as to be discharged to the accommodating space such that the air inside the accommodating space (S) flows in the entire accommodating space.

By this rotary air, the removal of foreign substances such as dust adhering to the hairs of a pet is facilitated, and efficient drying of moisture on the hairs of a pet after bathing is achieved.

The features described above by using the drawings are only the main features of the present invention, of which various designs are available within the technical scope thereof, and thus it is obvious that the present invention is not limited by the features of the drawings.

1. A pet healthcare apparatus with a bacteriostatic and sterilization system, in which an accommodating space (S) is formed to accommodate a pet, the pet healthcare apparatus is provided with a care device, wherein the care device comprises:
a lower device including a front member, a rear member, and a lateral member;
a bottom plate coupled to an upper portion of the lower device and having bottom lateral grooves at opposite lateral sides thereof;
discharge parts coupled to each of the bottom lateral grooves to enable purified air to be removed from the lower device and then supplied to the accommodating space (S); and
a suction part allowing air of the accommodating space to flow toward a case space part disposed inside the lower device,
wherein the lower device further comprises:
copper coating fin heaters configured to heat air passing therethrough;
a fan for circulating air;
a fan discharge part disposed in a lateral portion of the fan and through which air is discharged from the fan; and
a first guide and a second guide each configured to surround the fan and allowing air discharged from the fan to How therebetween,
wherein the suction part is made of an antibacterial material, and
wherein the suction part comprises:
a filter (P) collecting pet hairs and dust; and
a HEPA filter (H) collecting and antibacterializing fine dust, dust, fungal spores, and bacteria; and
a UV-C LED lamp (R) having a sterilizing function.

2. (canceled)

3. The pet healthcare apparatus with a bacteriostatic and sterilization system according to claim 1, wherein a first discharge hole and a second discharge hole are formed in the discharge part, the first discharge hole and the second discharge hole formed at double tilt angles of 5° and 13°.

4. The pet healthcare apparatus with a bacteriostatic and sterilization system according to claim 3, wherein
the first discharge hole and the second discharge hole are configured such that air is discharged toward a bottom plate due to one angle of the double tilt angles and air is discharged in a lateral direction of the accommodating space due to another angle of the double tilt angles.

5. (canceled)

6. The pet healthcare apparatus with a bacteriostatic and sterilization system according to claim 4, wherein air is swirled in one direction by a fan provided inside the lower device, and the swirled air is accelerated by the first discharge hole and the second discharge hole when the swirled air is provided to the accommodating space.

7. The pet healthcare apparatus with a bacteriostatic and sterilization system according to claim 1, further comprising a suction hole disposed in the front member of the lower device and allowing air outside the pet healthcare apparatus to flow into the case space part.

8. A pet healthcare apparatus with a bacteriostatic and sterilization system, in which an accommodating space (S) is formed to accommodate a pet, the pet healthcare apparatus is provided with a care device, wherein the care device comprises:
a lower device including a front member, a rear member, and a lateral member,
a bottom plate coupled to an upper portion of the lower device and having bottom lateral grooves formed at opposite lateral sides thereof;
discharge parts coupled to each of the bottom lateral grooves to enable purified air to be removed from the lower device and then supplied to the accommodating space (S); and
a suction part allowing air of the accommodating space to flow toward a case space part disposed inside the lower device,
wherein the suction part comprises:
a suction body;
a through-hole formed at an upper portion of the suction body;
a through-groove cover formed at the through-hole;
a filter (P) disposed at the through-hole;
a HEPA filter (H) installed at the discharge hole of the suction part; and
an UV-C LED lamp (R) mounted at a rear surface of the HEPA filter.

9. The pet healthcare apparatus with a bacteriostatic and sterilization system according to claim 8, wherein
a rear coupling groove is formed in the rear member and a suction protrusion is formed at a lower portion of the suction pan such that suction protrusion is coupled to the rear coupling groove to allow air to be supplied to the case space part therethrough.

10. (canceled)

11. The pet healthcare apparatus with a bacteriostatic and sterilization system according claim 9, wherein the suction part inch an air purifier having a plasma wave function generating oxygen ions.

12. The pet healthcare apparatus with a bacteriostatic and sterilization system according to claim 11, wherein the suction part further includes a deodorizing filter removing smell and odors from the inside of the pet healthcare apparatus.
